(19) United States
(12) Patent Application Publication
(10) Pub. No.: US 2019/0088365 A1
(43) Pub. Date: Mar. 21, 2019

(54) NEUROPSYCHOLOGICAL EVALUATION SCREENING SYSTEM

(71) Applicants: SENTIMETRIX, INC, Bethesda, MD (US); E-SURE NEUROPSYCHOLOGICAL R&D, LTD, Tel Aviv (IL)

(72) Inventors: Venkatramanan Siva SUBRAHMANIAN, Potomac, MD (US); Vadim KAGAN, Bethesda, MD (US); Alexander DEKHTYAR, San Louis Obispo, CA (US); Joshua Michael TERRELL, Santa Maria, CA (US); Andrew Charles STEVENS, Leesburg, VA (US); Ayala BLOCH, Ariel (IL)

(21) Appl. No.: 16/080,676

(22) PCT Filed: Mar. 1, 2017
(86) PCT No.: PCT/IL17/50264

§ 371 Date: Aug. 29, 2018

Related U.S. Application Data
(60) Provisional application No. 62/301,791, filed on Mar. 1, 2016.

(51) Int. Cl. G16H 50/20; G06N 20/10; G16H 10/20

(52) U.S. Cl. CPC G16H 50/20; G16H 10/20; G06N 20/10

(57) ABSTRACT

A system, method and software application for neuropsychological evaluation screening, the system comprising: an application server controlling a software application installed in a communication device, the server comprising at least one processor configured to execute code instructions for: generating a classifier engine based on communication data and diagnosis of diagnosed subjects by input feature signals of the communication data to a plurality of classifiers, calculate predictive accuracy for each classifier, and generate a combination of classifiers based on the predictive accuracy; collecting text and vocal data from multiple communication channels at the communication device; input feature signals of the collected data to the plurality of classifiers; and execute the combination of classifiers.

### FIELD OF THE INVENTION

The present disclosure generally relates to computerized neuropsychological screening, and more specifically to probabilistic prediction of neuropsychological deficits.

### BACKGROUND

Various forms of brain damage affect virtually every demographic group in societies around the world. Apart from traumatic injuries commonly incurred during automobile accidents, military activities, and sports, compromised brain function can also result directly or indirectly from diseases, medical interventions such as chemotherapy, and even aging.

Often dubbed an “invisible disability,” brain damage frequently shows few external signs despite having drastic cognitive, emotional, social, and behavioral effects. As such, despite the many advanced technologies available for detecting brain damage, many cases are never diagnosed—or diagnosed so late that the benefits of early medical treatment are not available to the patient. Moreover, even when a clear source of brain damage has been identified or is suspected, its often-dramatic effects on the daily life of the patient are commonly unrecognized or overlooked, though they may result, in significant individual and societal costs.

Neuropsychological assessment can shed light on the deficits associated with brain damage in the absence of clear cut external symptoms, provide guidelines for diagnosis and care, and predict functional potential and recovery. However, many individuals who could benefit from such evaluation are simply not referred to neuropsychologists. On one hand, as described above, the population requiring neuropsychological assessment is grossly underdiagnosed because healthcare professionals often do not have feasible and cost-effective tools to determine whether patients should be referred. On the other hand, assessment is a costly and time-consuming process that must be handled by trained specialists, such that HMOs and insurance companies are hesitant to fund assessments unless there is a clear cut need. The unfortunate result is that patients who require evaluation often do not receive it.

Aside from the obvious health-related, emotional, and social consequences for individuals who do not receive necessary care, the low referral rates have significant financial consequences at various levels. Early identification of brain damage symptoms can prevent unnecessary downstream healthcare costs stemming deterioration or the need for medical and psychological treatments. Accurate diagnosis can also lead to proper care, which can help patients get back into the workforce, with clear financial implications at both the personal and societal levels. There is therefore a critical need for a product that will help healthcare professionals and healthcare funding organizations to quickly, easily, and accurately decide which patients should be referred for neuropsychological assessment.

In this context, it has been known for almost 40 years that brain damage can lead to various forms of speech impairment, and recent research suggests that even milder forms of brain damage can have subtle but detectable effects on spoken communication.

### SUMMARY

Some embodiments of the present invention may provide a solution for making quick, easy, and accurate decision whether a patient should be referred for neuropsychological assessment. For example, some embodiments of the present invention utilize factors known to differentiate between individuals with and without neuropsychological deficits, such as different kinds of brain damage. Some embodiments provide a computer program that leverages symptoms correlated with brain damage in order to better identify and help affected patients.

Some embodiments of the present invention may provide a system for neuropsychological evaluation screening, the system comprising: an application server controlling a software application installed in a communication device, the server comprising at least one processor configured to execute code instructions for generating a classifier engine based on communication data and diagnosis of diagnosed subjects by input feature signals of the communication data to a plurality of classifiers, calculate predictive accuracy for each classifier, and generate a combination of classifiers based on the predictive accuracy; collecting text and vocal data from multiple communication channels at the communication device; input feature signals of the collected data to the plurality of classifiers; and execute the combination of classifiers.

In some embodiments of the present invention, the processor is configured to execute code instructions for receiving authorizations to collect data from particular channels.

In some embodiments of the present invention, the processor is configured to execute code instructions for performing signal extraction by calculating histogram values and generating a user feature vector by combining the histogram values.

In some embodiments of the present invention, generating a classifier engine is performed by generating subject feature vectors, feeding tuples into each of the classifiers, calculating predictive accuracy for each classifier and generating a probabilistic predictor engine.

In some embodiments of the present invention, the processor is configured to execute code instructions for obtaining predictions generated by each of the classifiers and calculating an overall probability that the prediction is correct.

Some embodiments of the present invention provide a method for neuropsychological evaluation screening, the method comprising: generating a classifier engine based on communication data and diagnosis of diagnosed subjects by input feature signals of the communication data to a plurality of classifiers, calculate predictive accuracy for each classifier, and generate a combination of classifiers based on the predictive accuracy; collecting text and vocal data from multiple communication channels at the communication device; inputting feature signals of the collected data to the plurality of classifiers; and executing the combination of classifiers.

In some embodiments of the present invention, the method comprises receiving authorizations to collect data from particular channels. In some embodiments of the present invention, the method comprises performing signal extraction by calculating histogram values and generating a user feature vector by combining the histogram values. In some embodiments of the present invention, generating a classifier engine is performed by generating subject feature vectors, feeding tuples into each of the classifiers, calculating predictive accuracy for each classifier and generating a probabilistic predictor engine. In some embodiments of the present invention, the method comprises obtaining predictions generated by each of the classifiers and calculating an overall probability that the prediction is correct.

### BRIEF DESCRIPTION OF THE DRAWINGS

Some non-limiting exemplary embodiments or features of the disclosed subject matter are illustrated in the following drawings.

In the drawings:

FIG. 1 is a schematic illustration of a system for neuropsychological evaluation screening, according to some embodiments of the present invention;

FIG. 2 is a schematic flowchart illustrating a method for neuropsychological evaluation screening, according to some embodiments of the present invention;

FIG. 3 is a schematic flowchart illustrating a method for signal data extraction, according to some embodiments of the present invention;

FIG. 4 is a schematic flowchart illustrating a method for creation of a classifier, according to some embodiments of the present invention;

FIG. 5 is a schematic flowchart illustrating a method for assessing probability of neuropsychological deficit in a patient user, according to some embodiments of the present invention.

With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Identical or duplicate or equivalent or similar structures, elements, or parts that appear in one or more drawings are generally labeled with the same reference numeral, optionally with an additional letter or letters to distinguish between similar entities or variants of entities, and may not be repeatedly labeled and/or described. References to previously presented elements are implied without necessarily further citing the drawing or description in which they appear.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

### DETAILED DESCRIPTION

Brain damage is a potential source of concern whenever any kind of head trauma occurs, and can also be caused by disease, medical intervention, and aging. Despite the often dramatic implications of brain damage, affected individuals are frequently left undiagnosed for extended periods of time, particularly when the damage is milder, indirect, or not attributed to a known injury.

Some embodiments of the present invention provide a system, a method and a software application for screening of neuropsychological deficits. A patient user may install the application on their device. By analyzing a host of voice- and text-based features present in their written and/or voice communications, the system may extract relevant textual and/or voice features and then apply relevant technical analysis of the resulting features to predict the probability that they are associated with impairments in various areas of cognitive, emotional, social, and behavioral functioning. This will statistically indicate the potential benefit of detailed neuropsychological assessment for the user, to help determine the course of further neuropsychological or medical evaluation.

Some embodiments of the present invention are suitable for multiple mobile phone platforms (e.g., iOS, Android) and multiple devices (e.g. iPad, tablets, laptops) that can be used by individuals to determine the probability that they need to seek further medical evaluation, as well as by doctors and other medical professionals or organizations that seek to help individuals who are at risk for or have known or suspected brain damage. Specifically, after monitoring the user's speech and writing patterns, the provided system may report the probability that such patterns are associated with impairments in various areas of cognitive, emotional, social, and behavioral functioning. The resulting profile constitutes a statistical indication of the potential benefit of detailed neuropsychological assessment for the user, which may be used by doctors alongside additional clinical considerations in determining the course of further neuropsychological or medical evaluation.

The provided system, method and software application, facilitate for a community of mental health professionals, doctors, insurance companies, and support groups to better identify individuals potentially suffering from the effects of brain damage, thereby increasing their access to the right assessment and treatment as fast as possible.

Some embodiments of the present invention may include a system, a method, and/or a computer program product. The computer program product may include a tangible non-transitory computer readable storage medium (or media) having computer readable program instructions thereon for causing a processor to carry out aspects of the present invention. Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including any object oriented programming language and/or conventional procedural programming languages.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The system and method provided by some embodiments of the present invention may enable assessment of a subject in order to detect indications of suspected brain damage. Such assessment may assist a user in deciding whether to seek a more precise examination, for example if the assessment provides indications of brain damage.

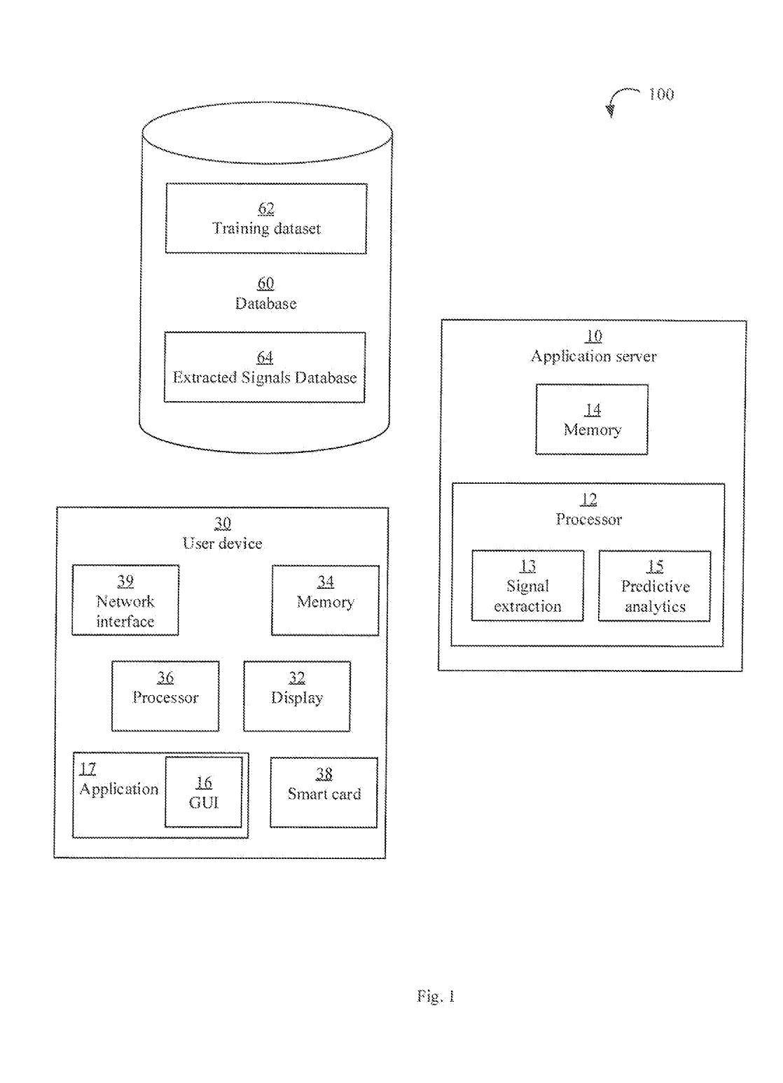

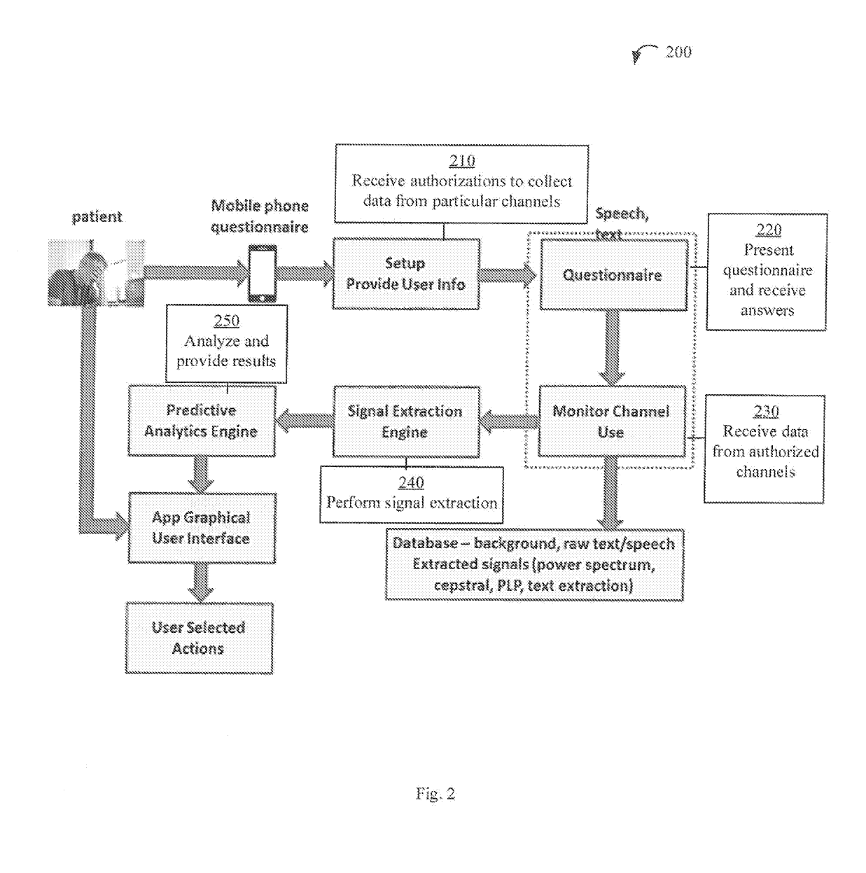

Reference is now made to FIG. 1, which is a schematic illustration of a system 100 for neuropsychological evaluation screening, according to some embodiments of the present invention. Further reference is made to FIG. 2, which is a schematic flowchart illustrating a method 200 for neuropsychological evaluation screening, according to some embodiments of the present invention.

System 100 may include an application server 10. Application server 10 may store and/or manage a software application 17, which may be downloaded by a user to a user device 30, for example via an application store and/or from application server 10. User device 30 may be a computing device such as, for example, a desktop computer, a mobile device, a laptop, a tablet computer, a cellular device, a smartphone, and/or any other suitable computing device. User device 30 may include a display 32, a memory 34, a processor 36, a smart card 38, and at least one network interface 39. User device 30 may communicate with application server 10, for example by network interface 39. Application server 10 may control and/or communicate with user device 30 by the downloaded application 17, which may include a graphical user interface (GUI) 16.

Application server 10 may include at least one processor 12 and a non-transitory memory 14. Memory 14 may store code instructions executable by processor 12. When executed by processor 12, the code instructions may cause processor 12 to carry out the methods described herein, for example method 200.

In some embodiments of the present invention, processor 12 creates a database 60 that includes data to facilitate the neuropsychological evaluation screening. For example, Processor 12 may execute a predictive analytics classifier engine 15 to classify extracted signal as implying a neuropsychological deficit, and/or which neuropsychological deficit is implied.

In some embodiments of the present invention, application 17 may have patient users, therapist users and third party users. Third party users may be, for example, representatives of an insurance company and/or insurance agents. Therapist users may be psychologists or other therapists, medical professionals, support group members/directors and/or clinic or hospital personals. Patient users use application 17 for assessment the probability of a neuropsychological deficit in themselves.

In some embodiments of the present invention, GUI 16 may require and/or obtain indication by a user of device 30 about whether they are patient users, therapist users and/or third party users.

As indicated in block 210, processor 12 may receive by GUI 16 instructions and/or authorizations to collect data from particular channels, such as from functionalities of device 30 and/or from software applications installed in device 30. In some embodiments of the present invention, once application 17 is downloaded to device 30, GUI 16 may enable a patient user to set a patient user profile, settings and/or definitions of application 17, including authorizations to monitor data. For example, processor 12 may receive authorization to collect textual data from e-mail, short messaging service and/or other messaging applications. For example, processor 12 may receive authorization to collect voice data, for example from telephone conversation and/or other vocal conversation applications.

For example, in some embodiments of the present invention, GUI 16 may include a setup page presenting to a patient user a plurality of optional channels, i.e. messaging and/or vocal communication applications through which processor 12 may gather textual and/or vocal signals generated by the patient user. The patient user may select via the setup page authorized channels, i.e. channels in which the patient user authorizes processor 12 to gather the data. For example, the patient user may cheek boxes next to respective channel names, in order to authorize the use of the respective channels.

In some embodiments of the present invention, processor 12 may obtain data from an authorized channel by receiving from a user login information and/or logging into a respective account in a respective application server. In some embodiments of the present invention, processor 12 may obtain data from an authorized channel by receiving from a user authorization to monitor keystrokes on user device 30, for example when an authorized channel and/or application 17 is active.

In some embodiments of the present invention, application 17 may trigger device 30 to send textual and/or vocal data gathered from authorized channels, for example every pre-determined period of time, for example every two hours, and/or upon gathering of new data.

In some embodiments of the present invention, GUI 16 may require a user to enter personal information such as, for example, a screen-name, age or age range, gender, zip code, health insurance details. In some embodiments of the present invention, application 17 may retrain from accessing identifying information, such as phone number, nor any other unauthorized information which may be stored on device 30.

In some embodiments of the present invention, GUI 16 may include a legal agreement page, on which a legally binding electronic disclaimer, waiver, and/or consent form may be presented and lay out the terms and conditions of engagement with the subject. GUI 16 may require an indication of consent from the user before monitoring of channels by application 17 is activated.

In some embodiments of the present invention, GUI 16 may require a therapist user to indicate personal administrative information such as, for example, name, phone number and/or address. GUI 16 may include a page upon which a therapist user may indicate alert conditions, e.g. conditions under which they authorize processor 12 to connect them with a patient user. For example, a therapist user may authorize processor 12 to connect them with a patient user that requests to be connected with a therapist user and is within a specified location range. For example, a therapist user may authorize processor 12 to connect them with a patient user within a specified location range that have any or a particular type of neuropsychological deficit with high probability according to the calculations of processor 12, e.g. with probability above a predefined threshold.

In some embodiments of the present invention, GUI 16 may require a third party user to indicate personal administrative information such as, for example, name, phone number, address, the third party's name and/or a geographical region of interest. GUI 16 may present to third party users anonymous statistical information obtained based on obtained data about the patient users. For example, a third party representative may define by GUI 16 which statistical information is presented to him. For example, GUI 16 may present and/or be configured by the representative to present information such as percentage of patient users in the indicated region of interest with probability for certain categories of deficits over a certain probability threshold, or percentage of patient users insured by the third party with probability for certain categories of deficits over a certain probability threshold, or percentage of patient users in the indicated region of interest insured by the third party with probability for certain categories of deficits over a certain probability threshold, or any other suitable information configuration.

As indicated in block 220, processor 12 may present via device 30 a questionnaire, for example by GUI 16. GUI 16 may enable filling of the questionnaire by a patient user. For example, by the questionnaire, processor 12 may receive from device 30 patient user information about events and or symptoms indicative about suspected brain damage such as, for example, experiences that could potentially have led to brain damage and/or indications known to be associated with brain damage.

GUI 16 may include textual, vocal and/or choice questionnaire items, for example questions which may require and/or enable input of answers by writing, speaking, recording, checking and/or clicking. For example, questionnaire questions may include questions about past injuries, infections, medical interventions and/or other events that may involve and/or affect the brain. For example, questionnaire questions may include questions about current medical condition and/or treatment. For example, questionnaire questions may include questions about vocational, social, cognitive, behavioral, and/or emotional changes in daily functioning. For example, questionnaire questions may require a patient user to tell about themselves and/or about how they feel. The text, voice and/or other answer inputs and/or extracted values are stored by processor 12.

In some embodiments of the present invention, GUI 16 may convert vocal input of the patient user to text. In some embodiments, GUI 16 may present the converted input to the patient user and enable the patient user to edit the text. The converted input and/or the edited converted input is stored as answer input by processor 12, for example along with the vocal input.

Processor 12 may store the questionnaire answers of each patient user in a dedicated storage partition in database 60, in a log table that may include, for example, along with answers input, a respective user name, time of input and/or location of device 30.

As indicated in block 230, processor 12 may receive by application 17 data from device 30 from the authorized channels, such as textual data and/or voice data from various from functionalities and/or software applications, according to the received authorizations, For example, processor 12 may monitor device 30 via application 17, which may collect data from device 30 and/or push the data to processor 12, for example periodically, continuously and/or upon collection of new data. For example, processor 12 may receive data from the authorized channels whenever an authorized channel is activated and/or used in user device 30.

In some embodiments of the present invention, processor 12 creates a database 60, in which signal data associated with patient users and/or user devices is stored in corresponding folders. For example, each user device 30 may have a corresponding extracted signal database 64 in database 60. Accordingly, database 60 may include multiple extracted signal database 64, each for another user device 30.

In some embodiment of the present invention, processor 12 may perform session of gathering signal data from authorized channels, for example by logging in to application accounts authorized by the patient user. For example, processor 12 may perform a gathering session periodically, continuously, upon generation of new data or upon the patient user's command. In some embodiments, processor 12 may store the data gathered in a session along with a respective timestamp and/or for example, in a separate storage partition in a corresponding extracted signal database 64. For example, GUI 16 may include a button by which a patient user may send a request to processor 12 to perform a gathering session immediately, and processor 12 may receive such commend and perform a gathering session upon receiving the command. This may enable a user to receive analysis of a data gathered in a specific time, for example when the patient user suspects that they produced relevant content in an authorized channel and/or when the patient user wishes a current assessment by application 17. In some embodiments, GUI 16 may enable a patient user to turn off monitoring and/or suspend or cancel authorization to monitor a certain channel. When monitoring is turned off for a channel, no new data is collected from the channel.

In some embodiments of the present invention, processor 12 may monitor keystrokes pressed on device 30, for example only when an authorized channel and/or application is active. For example, processor 12 may monitor by application 17 when a “return,” “submit,” “post” or “send” button is pressed. Accordingly, for example, processor 12 may store in a corresponding extracted signal database 64 a log of the keystroke data together with corresponding timestamps.

Timestamps allocated to data stored in database 64 may be used by processor 12, for example, to record which data went through processing and which data is new data, e.g. data before processing. For example, upon completing to process data stored with relation to a certain timestamp and/or data in a corresponding storage partition, processor 12 may flag the certain timestamp and/or corresponding storage partition as processed. Data flagged as processed may be pushed to application 17 at device 30 to enable presentation of the processed data to the user.

As indicated in block 240, processor 12 may perform signal extraction, for example execute a signal extraction engine 13. In some embodiments of the present invention, processor 12 store every predefined period, for example every day or a predefined number of hours, a set of extracted text and/or vocal signals, for example in a dedicated storage partition in extracted signal database 64.

Reference is now made to FIG. 3, which is a schematic flowchart illustrating a method 300 for signal data extraction, according to some embodiments of the present invention. For example, method 300 may be performed by processor 12 by executing signal extraction engine 13.

As indicated in block 310, processor 12 may collect textual and speech interactions of a patient user U via authorized channels within a given duration D, for example a given day. Processor 12 may combine the textual interactions of user U in a duration D into a single body of text Text(U,D). Processor 12 may combine the speech interactions of user U in duration D to a single body of speech Speech(U,D) associated with that specific user on that specific day. From each of Text(U,D) and Speech(U,D), we will extract a suite of signals associated with user U and duration D, as described in more detail herein.

As indicated in block 320, processor 12 may extract signals from Text(U,D) and Speech(U,D) by calculating histogram values, each corresponding to another feature type of the collected data. For example, processor 12 may extract from Speech(U,D) signals of Power Spectrum Features (PSF), Cepstral Features (CF), Perceptual Linear Prediction (PLP)-Related Features (PRF) and any other suitable speech data features.

In order to extract a PSF signal, processor 12 may divide Speech(U,D) to N segments (i.e. time duration intervals) of a predetermined duration, for example of 10 milliseconds each or any other suitable duration. For each of the N segments, processor 12 calculates the average sound frequency of the speech included in the segment. Processor 12 may generate a histogram of about 256 bins of frequency ranges dividing a sound power spectrum. For example, for each bin corresponding to a frequency range [X,Y] (e.g. X to Y Khz), processor 12 may calculate a histogram value Bin(X,Y), which is the percentage of segments out of the N segments whose calculated average frequency or energy falls within the [X,Y] frequency interval. For example:

$$Bin ( X , Y ) = \frac{\vert \{ \begin{matrix}i \text{\textbar} i is a 10 ms interval in Speech ( U , D ) s . t . X \leq \\ AvgFreq ( i ) \leq U\end{matrix} \} \vert}{\vert \{ j \text{\textbar} j is a 10 ms interval in Speech ( U , D ) \} \vert} ,$$

wherein Bin(X,Y) is a probability that reflects the probability that a given speech interval from Speech(U,D) has an average frequency larger than or equal to X and smaller than or equal to Y. Alternative histogram values may be, for example:

BinEnergy(X,Y)=avg{energy(i)|i is a 10 ms interval in Speech(U,D)s.t.X≤AvgFreq(i)≤U}.

wherein the energy histogram is storing average energy within a given frequency interval, capturing the intensity of the signal within that frequency range.

In some embodiments, pitch, amplitude, and/or other wave-form related signatures can likewise be converted into histograms and stored as signals. For instance for pitch histogram:

BinPitch(X,Y)=avg{pitch(i)|i is a 10 ms interval in Speech(U,D)s.t.X≤AvgFreq(i)≤U}.

It will be appreciated that BinAmplitude(X,Y) for amplitude histogram and other relevant speech quantities can be similarly calculated and stored.

In order to calculate a CF signal, processor 12 may associate histograms with each frequency range [X,Y] that are derived from cepstral data. For instance, for each such interval, processor 12 may identify the average rate of change of frequencies in the range [X,Y] across all the segments of the N segments whose average frequency lies within the [X,Y] interval.

In order to calculate a PRF signal, processor 12 may execute methods of the psycho-physics of hearing via spectral resolution, intensity, and loudness in conjunction with a pole model in order to approximate the auditory spectrum, i.e. may approximate the impact of a voice signal on the human auditory system (i.e. how it is heard as opposed to how the signal is generated). Thus, processor 12 may generate a set of cepstral coefficients, for example by standard methods, which may be used by processor 12 as parameters in the predictive model of classifier engine 15.

Processor 12 may extract signals, e.g. histogram values, from Text(U,D). For example, processor 12 may extract from Text(U,D) emotion representative data detected according to textual emotion detection methods, and generate from the emotion representative data emotion signals by calculation of histograms. Processor 12 may divide Text(U,D) to N segments (i.e. corresponding to time duration intervals) of a predetermined duration, for example of 10 milliseconds each or any other suitable duration. For example, each segment may include text segment typed within the corresponding duration. For example, for each of the N segments, processor 12 computes an intensity of depression/fear/anxiety/anger in that textual segment using known methods as well as related emotion extraction methods.

Processor 12 may calculate an average value Text(U,D,E,i), e.g. the average strength S of emotion E in a one of the N segments of Text(U,D), e.g. a segment i, of the text body Text(U,D) associated with subject U on duration D. Based on the average value Text(U,D,E,i), processor 12 may compute an emotion histogram for each user U and emotion E. For example, for an interval [W,Z] of the intensity of emotion E, processor 12 may calculate an emotion histogram value BinEmotionₑ(W,Z):

$${BinEmotion}_{e} ( W , Z ) = \frac{\vert \{ \begin{matrix}i \text{\textbar} i is a 10 ms time slice in Speech ( U , D ) \& W \leq \\ {intensity}_{ɛ} ( i ) \leq Z\end{matrix} \} \vert}{total number of 10 ms time slice in Speech ( U , D )} .$$

Note that one such histogram can be defined by processor 12 for each emotion E that is detected by an existing text-based emotion extraction engine. In the above formula, intensityₑ(i) denotes that average intensity of emotion E detected in segment I, for example by an existing emotion detection engine.

As indicated in block 330, processor 12 may generate a segment feature vector Vd(U,D) associated with user U on duration D, by concatenating the calculated histogram values associated with user U on duration D into one vector. It is noted that any histogram can be viewed as a vector. For instance, an histogram associated with an emotion (e.g. depression), wherein the intensities of this emotion are recorded on a 0 to 1 scale, may be calculated over different [A,B] intensity intervals such as [0,0.1], [0.1,0.2], . . . ,[0.9,1]. In this case, a vector associated with this emotion is the vector:

<BinEmotionₑ(0.0.1), BinEmotionₑ(0.1,0.2), . . . , BinEmotionₑ(0.9,1)>.

Similarly, processor 12 may generate a user feature vector Vm(U,M) associated with a patient user U across a period of time M consisting of multiple duration segments D, such as a month, for example by averaging the value of each component of Vd(U,D) over all the duration segments, for example the one day segments in the one month period.

In some embodiments, processor 12 may generate a classified training dataset 62 for the evaluation. For example, training dataset 62 may include positive data extracted from subjects with diagnosed neuropsychological deficits and negative data extracted from patients with no diagnosed neuropsychological deficits. Positive data may be stored in database 60 sorted or tagged according to multiple categories, for example according to different neuropsychological deficits of the corresponding subjects. For example, data may be marked by processor 12 with positive or negative markings for specific neuropsychological categories based on the corresponding subject's performance in neuropsychological measures of these categories.

In some embodiments, processor 12 may create a classifier engine 15 based on training dataset 62. For example, processor 12 may extract signals from test subjects, i.e. from identified test user devices used by subjects with pre-diagnosed neuropsychological deficits. In some embodiments, the test subjects may include subjects that have no diagnosed neuropsychological deficits. For each of the test user devices, processor 12 may perform signal extraction and store the signals extracted within a predefined period, for example in a storage partition respective to the test subject and/or the period of time. For example, in some embodiments of the present invention, each test subject may use application 17 by a device 30 over a period of time, for example of a certain number of days. On each pre-defined time-period, for example a day, signal extraction engine 13 may store the extracted signals and their features in a dedicated storage partition. For example, for N test subjects and D days, processor 12 may generate a total of N*D rows of signals and respective feature data, along with corresponding positive and/or negative tagging with relation to diagnosed neuropsychological deficits.

The extracted textual and/or vocal signals may be marked according to the pre-diagnosed deficits of the respective test subjects and stored with corresponding marks and/or tags in training dataset 62. In order to create classifier 15, processor 12 may analyze parameters of the training dataset 62 to formulate decision functions and/or a classifier model that may constitute classifier engine 15. Accordingly, processor 12 may use test subjected in order to train and/or create classifier engine 15, for example by learning a predictive model from data obtained from positively and/or negatively diagnosed subjects.

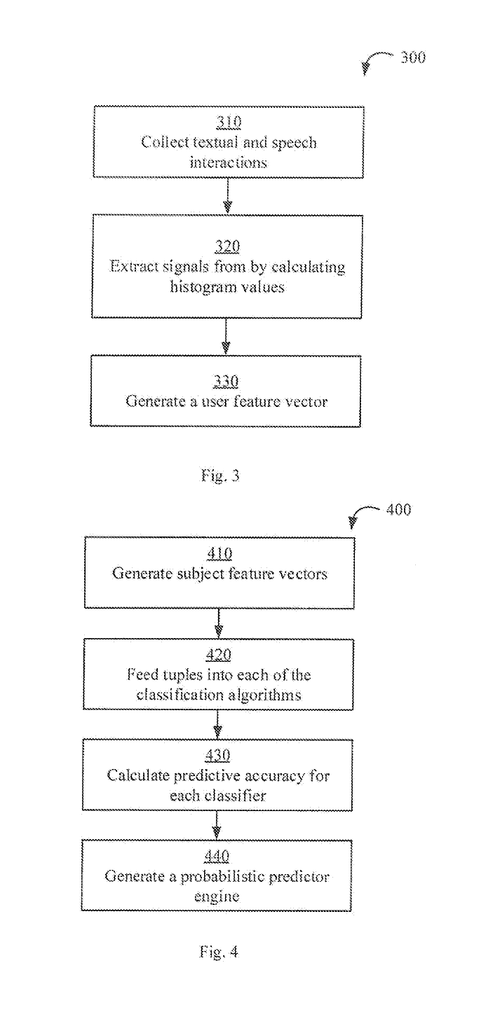

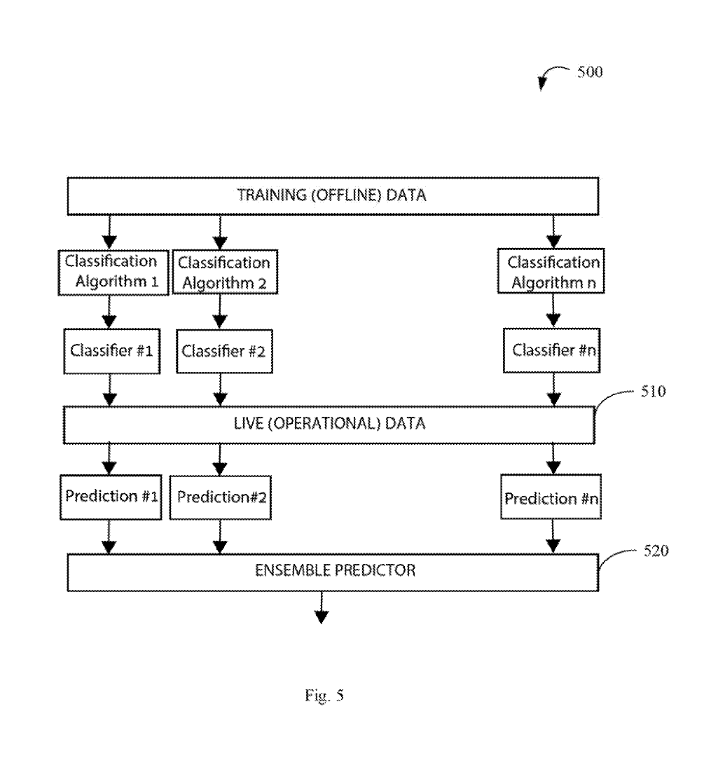

Reference is now made to FIG. 4, which is a schematic flowchart illustrating method 400 for creation of classifier 15. As indicated in block 410, processor 12 may generate, as described in detail herein, subject feature vectors that may be annotated f₁, f₂, . . . , fₙ. Each feature vector is associated with a subject sᵢ and contains the signal data collected about that subject and aggregated over some fixed period of time (e.g. one interview session or one day or one week). In some embodiments, each or some of the collected textual and speech data is also reviewed by a clinical psychologist with expertise in brain damage. Based on their analysis, each subject sᵢ has an assessment result aᵢ which is set to 1 if the assessment is positive and to 0 if the assessment is negative. For example, a positive assessment is given when a therapist, for example a clinical psychologist, indicates that there is high probability that the subject has a neuropsychological deficit. Additionally or as an alternative, processor 12 may receive from a therapist user, and/or may set an additional secondary dependent variable, an indication whether an assessment given by processor 12 match an assessment given by the therapist user. Accordingly, processor 12 may generate a subject training set as shown herein as Table 1. As shown in Table 1, the subject training set stores a tuple (fₙ,aₙ) for each Subject Sₙ.

TABLE 1

| | Feature Vector | |
|---|---|---|
| Subject | (bunch of columns) | Assessment |
| s₁ | f₁ | a₁ |
| s₂ | f₂ | a₂ |
| s₃ | f₃ | a₃ |
| . . . | . . . | . . . |
| . . . | . . . | . . . |

The feature vector column in Table 1 may include a set of N columns where N is the total number of features in the feature vector.

Then, processor 12 may initiate generation of classifier engine 15, for example by various classification algorithms, by learning conditions on parameters of the feature vectors which characterize positive subjects, i.e. cases when the assessment is a 1 (i.e. subject should see a medical professional for possible undiagnosed brain damage) and/or differentiate positive subjects from negative subjects, i.e. subjects for which the assessment is a 0. The classification algorithms may include, for example, decision trees, support vector machines, restricted Boltzmann machines, naïve Bayes classifiers, AdaBoost and/or Gradient Boost. Since the various classification algorithms have different benefits in different situations, processor 12 may generate engine 15 by creating a probabilistic predictor engine that merges the best of many different existing classifiers.

For example, as indicated in block 420, processor 12 may feed tuples {(f₁,a₁), . . . , (fₖ,aₖ)} into each of the classification algorithms, each generating a different classifier engine (i.e. classifier). Thus, if there are n classification algorithms, processor 12 generates n classifiers CL₁, . . . , CLₙ. As indicated in block 430, processor 12 may calculate predictive accuracy Acc(CLᵢ) for each of the generated classifiers CLₙ, wherein the predictive accuracy indicates a probability that the prediction made by classifier CLi is correct. For example, the predictive accuracy may include Accuracy, F1-measure, Mathews Correlation Coefficient, and/or any other suitable accuracy measure. For example, in case classifier CLi decides that a particular subject needs a neurophysiological screening, then Acc(CLᵢ) gives us the probability that this prediction is correct.

As indicated in block 440, processor 12 may generate a probabilistic predictor engine, used by classifier engine 15, configured to combine the classifiers predictions into a combined prediction, and combines the predictive accuracies of the different classifiers in order to calculate a probability that the combined prediction is correct.

As indicated in block 250, processor 12 may analyze signal data extracted from a device 30 of a patient user, for example a patient user who is not a test subject and/or is not pre-identified as negative or positive for neuropsychological deficits. As discussed herein, the extracted signal data is stored in extracted signal database 64. Processor 12 may feed data stored in database 64 with relation to a specific patient user as input to execute predictive analytics classifier engine 15. By classifier engine 15, processor 12 may decide whether the respective patient user is suspected as suffering from a neuropsychological deficit, and/or what neuropsychological deficit(s) they suffer from. For example, processor 12 may output a probabilistic value corresponding to the measure in which the parameters of the signal data of the respective patient user imply a neuropsychological deficit, and/or what neuropsychological deficit(s) they imply. The output provided by processor 12 may enable a patient user to decide, for example, whether a further detailed assessment is required.

Reference is now made to FIG. 5, which is a schematic flowchart illustrating method 500 for assessing probability of neuropsychological deficits in a patient user. For example, processor may calculate a patient feature vector nfᵢ of a patient user, associated with textual and speech communications of the patient user over some period of time. As indicated in block 510, processor 12 may use each of the classifiers CLⱼ on a feature vector nfᵢ to predict an assessment probabilities CLⱼ(nfᵢ), i.e. a predictions made about whether the patient user should go for an assessment or not by each individual classifier. Processor 12 may obtain the predictions CL₁(nfᵢ),CL₂(nfᵢ), . . . , CLₘ(nfᵢ) generated by each of the classifiers.

As indicated in block 520, processor 12 may calculate an overall probability that the prediction is correct. In some embodiments, for example, the probability may be calculated as follows, wherein Prob(nfᵢ should visit psychologist) is the probability that a patient user should visit a therapist to assess a neuropsychological condition.

$$Prob ( {nf}_{i} should visit psychologist ) = \frac{{\sum }_{{CL}_{j} ( {nf}_{i} ) = 1} Acc ( {CL}_{j} )}{{\sum }_{{CL}_{j} ( {nf}_{i} ) = 1} Acc ( {CL}_{j} )} .$$

The numerator of this expression captures the sum of the accuracies of those individual classifiers that stated that the subject needs a detailed neuropsychological assessment, while the denominator sums up the accuracy of all the classifiers. It is noted that probability measure can vary depending upon which measure of predictive accuracy Ace is actually used. For instance, F-measure standard accuracy vs. F-measure vs. Matthews correlation coefficient all give different results. Thus, in some embodiments, processor 12 may present by GUI 16 multiple prediction values of whether the patient user should visit a therapist to assess a neuropsychological condition.

Processor 12 may present to the patient user results, e.g. predictions of each or some of the individual classifiers, as well as the probability, for example according to one or more measures of predictive accuracy, that the patient user should visit a therapist.

In some embodiments of the present invention, processor 12 may present by GUI 16, for example along with the results, a list of potential medical personnel registered with application 17.

In some embodiments of the present invention, processor 12 may present by GUI 16, for example along with the results, a list of medical personnel available through a corresponding insurance company.

In some embodiments of the present invention, processor 12 may present by GUI 16, for example along with the results, a list of support groups that might be relevant.

In some embodiments of the present invention, processor 12 may present by GUI 16, for example along with the results, relevant actions for the user to take. In some embodiments, GUI 16 may enable a user to click on a button to automatically execute the action, for example communicate with the selected entity.

In some embodiments of the present invention, for example based on authorization from a patient user, processor 12 may contact a therapist user, a medical professional, a therapist and/or another third entity and/or to send the output value and/or identified suspected neuropsychological deficit to the third entity. The third entity may include, for example, a medical professional, a therapist, a doctor, a psychologist, a hospital, a support group, an insurance company and/or any other suitable entity. In some embodiments of the present invention, a therapist that treats the patient user, for example, regularly, is a therapist user of application 17 and/or provides a feedback to processor 12, for example by application 17 and/or a complementary application. For example, a therapist may receive the output value and provide a feedback regarding the accuracy of the assessment by processor 12, or a feedback including a diagnosis of the patient user, or indication about the patient user's lack of diagnosed deficit. Processor 12 may use the feedback to further train classifier engine 15. For example, processor 12 may use the diagnosed patient user as a test subject, thus, for example, updating classifier engine 15 and improving its predictive accuracy according to feedback from real therapists about patient users of application 17.

In some embodiments of the present invention, a participating medical provider who sees a subject sᵢ and assesses the person clinically, either through just an interview or through a more substantive assessment, can use application server 10 to interface to both learn more about the subject as well as provide feedback to application server 10.

In some embodiments of the present invention, processor 12 may calculate and/or present by GUI 16 a visualization of how a specific feature f changed over time in the patient user's extracted data. In some embodiments, processor 12 may provide and/or display a list of features selected by a user and/or a selected feature's values as a graph plotted against time. For instance, if the feature f of interest is a measure of the patient user's level of depression, GUI 16 may present a lime series graph plotting the patient user's intensity of depression, for example shown on the y-axis on a [0,1] scale, and/or time plotted on the x-axis.

In some embodiments, GUI 16 may enable a user to provide feedback. For example, GUI may include a button to click to confirm that this patient user required or didn't require an assessment. Processor 12 may then add the corresponding tuple, (nfᵢ, 1) or (nfᵢ, 0), to the training set.

In the context of some embodiments of the present disclosure, by way of example and without limiting, terms such as ‘operating’ or ‘executing’ imply also capabilities, such as ‘operable’ or ‘executable’, respectively.

Conjugated terms such as, by way of example, ‘a thing property’ implies a property of the thing, unless otherwise clearly evident from the context thereof.

The terms ‘processor’ or ‘computer’, or system thereof, are used herein as ordinary context of the art, such as a general purpose processor, or a portable device such as a smart phone or a tablet computer, or a micro-processor, or a RISC processor, or a DSP, possibly comprising additional elements such as memory or communication ports. Optionally or additionally, the terms ‘processor’ or ‘computer’ or derivatives thereof denote an apparatus that is capable of carrying out a provided or an incorporated program and/or is capable of controlling and/or accessing data storage apparatus and/or other apparatus such as input and output ports. The terms ‘processor’ or ‘computer’ denote also a plurality of processors or computers connected, and/or linked and/or otherwise communicating, possibly sharing one or more other resources such as a memory.

The terms ‘software’, ‘program’, ‘software procedure’ or ‘procedure’ or ‘software code’ or ‘code’ or ‘application’ may be used interchangeably according to the context thereof, and denote one or more instructions or directives or electronic circuitry for performing a sequence of operations that generally represent an algorithm and/or other process or method. The program is stored in or on a medium such as RAM, ROM, or disk, or embedded in a circuitry accessible and executable by an apparatus such as a processor or other circuitry. The processor and program may constitute the same apparatus, at least partially, such as an array of electronic gates, such as FPGA or ASIC, designed to perform a programmed sequence of operations, optionally comprising or linked with a processor or other circuitry.

The term ‘configuring’ and/or ‘adapting’ for an objective, or a variation thereof, implies using at least a software and/or electronic circuit and/or auxiliary apparatus designed and/or implemented and/or operable or operative to achieve the objective.

A device storing and/or comprising a program and/or data constitutes an article of manufacture. Unless otherwise specified, the program and/or data are stored in or on a non-transitory medium.

In case electrical or electronic equipment is disclosed it is assumed that an appropriate power supply is used for the operation thereof.

The flowchart and block diagrams illustrate architecture, functionality or an operation of possible implementations of systems, methods and computer program products according to various embodiments of the present disclosed subject matter. In this regard, each block in the flowchart or block diagrams may represent a module, segment, or portion of program code, which comprises one or more executable instructions for implementing the specified logical function(s). It should also be noted that, in some alternative implementations, illustrated or described operations may occur in a different order or in combination or as concurrent operations instead of sequential operations to achieve the same or equivalent effect.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprising”, “including” and/or “having” and other conjugations of these terms, when used in this specification, specify the presence of stated features, integers, steps, operations, elements, and/or components, but do not preclude the presence or addition of one or more other features, integers, steps, operations, elements, components, and/or groups thereof.

The terminology used herein should not be understood as limiting, unless otherwise specified, and is for the purpose of describing particular embodiments only and is not intended to be limiting of the disclosed subject matter. While certain embodiments of the disclosed subject matter have been illustrated and described, it will be clear that the disclosure is not limited to the embodiments described herein. Numerous modifications, changes, variations, substitutions and equivalents are not precluded.

What is claimed is:

1. A system for neuropsychological evaluation screening, the system comprising:
an application server controlling a software application installed in a communication device, the server comprising at least one processor configured to execute code instructions for:
generating a classifier engine based on communication data and diagnosis of diagnosed subjects by input feature signals of the communication data to a plurality of classifiers, calculate predictive accuracy for each classifier, and generate a combination of classifiers based on the predictive accuracy;
collecting text and vocal data from multiple communication channels at the communication device;
inputting feature signals of the collected data to the plurality of classifiers; and
executing the combination of classifiers.

2. The system of claim 1, wherein the processor is configured to execute code instructions for receiving authorizations to collect data from particular channels.

3. The system of claim 1, wherein the processor is configured to execute code instructions for performing signal extraction by calculating histogram values and generating a user feature vector by combining the histogram values.

4. The system of claim 1, wherein generating a classifier engine is performed by generating subject feature vectors, feeding tuples into each of the classifiers, calculating predictive accuracy for each classifier and generating a probabilistic predictor engine.

5. The system of claim 1, wherein the processor is configured to execute code instructions for obtaining predictions generated by each of the classifiers and calculating an overall probability that the prediction is correct.

6. A method for neuropsychological evaluation screening, the method comprising:
generating a classifier engine based on communication data and diagnosis of diagnosed subjects by input feature signals of the communication data to a plurality of classifiers, calculate predictive accuracy for each classifier, and generate a combination of classifiers based on the predictive accuracy;
collecting text and vocal data from multiple communication channels at the communication device;
inputting feature signals of the collected data to the plurality of classifiers; and
executing the combination of classifiers.

7. The method of claim 6, comprising receiving authorizations to collect data from particular channels.

8. The method of claim 6, comprising performing signal extraction by calculating histogram values and generating a user feature vector by combining the histogram values.

9. The method of claim 6, wherein generating a classifier engine is performed by generating subject feature vectors, feeding tuples into each of the classifiers, calculating predictive accuracy for each classifier and generating a probabilistic predictor engine.

10. The method of claim 6, comprising obtaining predictions generated by each of the classifiers and calculating an overall probability that the prediction is correct.
